(19) United States
(12) Patent Application Publication
(10) Pub. No.: US 2016/0041178 A1
(43) Pub. Date: Feb. 11, 2016

(54) ARRAY-BASED PROXIMITY LIGATION ASSOCIATION ASSAYS

(71) Applicant: RAYBIOTECH, INC., Norcross, GA (US)

(72) Inventor: Ruo-Pan Huang, Norcross, GA (US)

(21) Appl. No.: 14/922,433

(22) Filed: Oct. 26, 2015

Related U.S. Application Data
(63) Continuation of application No. 13/509,721, filed on May 14, 2012.
(60) Provisional application No. 61/262,170, filed on Nov. 18, 2009.

(51) Int. Cl. G01N 33/58

(52) U.S. Cl. CPC G01N 33/582; G01N 33/58; G01N 2458/10

(57) ABSTRACT

Embodiments of this disclosure encompass methods, systems and probes for the detection of a target analyte in a sample. The method uses of the detection of at least two distinct sites on an analyte molecule, or the pairing of two distinct sites on two adjacent and contacting molecules, the sites being integral to the structure of a single molecule or positioned near one another due to the three-dimensional structure of the polypeptide. At least one of the detectable sites may be formed by a modification of a larger molecule. The methods of detecting a target analyte comprise contacting a sample with a pair of probes, each probe comprising a binding moiety capable of specifically binding to a target analyte o a tag thereon, and an oligonucleotide tail that comprises a PCR initiator region proximal to the target analyte binding moiety, a barcoding region uniquely associated with the target analyte binding moiety, and a connector-hybridizing region complementary to a region of a connector oligonucleotide; hybridizing a connector oligonucleotide to the connector-hybridizing regions of the probes and ligating the connector-hybridizing regions of the probes; PCR amplifying the ligated oligonucleotide tails; hybridizing the amplification product with a substrate-immobilized oligonucleotide that as regions complementary to the barcoding regions of the probes; digesting any single-strand DNA molecule; hybridizing a signaling oligonucleotide to the product of the previous step; and detecting the signal, thereby detecting the presence of the analyte in the sample.

### CROSS-REFERENCE TO RELATED APPLICATIONS

This application claims priority to U.S. Provisional Patent Application Ser. No. 61/262,170, entitled “ARRAY-BASED PROXIMITY LIGATION ASSOCIATION ASSAYS” filed on Nov. 18, 2009, the entirety of which is hereby incorporated by reference.

### TECHNICAL FIELD

The present disclosure is generally related to methods of detecting molecular associations between polypeptides and other molecules. The methods further relate to array-based systems.

### BACKGROUND

Enzyme-Linked ImmunoSorbent Assay (ELISA) has been routinely used in the lab for cytokine detection and quantification for over 40 years. In this method, a target protein is first immobilized to a solid support. The immobilized protein is complexed with an antibody that is linked to an enzyme and detected by incubating this enzyme-complex with a substrate that produces a detectable signal, the intensity of which can be proportional to the target protein concentration and used for protein quantification through parallel internal standard controls. If the target protein is captured through binding to its immobilized specific capture antibody, a sandwich ELISA assay is performed. Sandwich ELISA uses a pair of protein specific antibodies, greatly increasing the assay detection specificity and sensitivity.

Proximity ligation assay (PLA) is a recently developed method in which specific proteins are analyzed by converting detection reactions to DNA sequences. In this method target molecules are recognized by two or more proximity probes that are prepared by attaching DNA strands to affinity binders. When three of such probes bind to a common target molecule/molecules, the free ends of two of the probes are brought in proximity and are capable of hybridizing, at some distance from each other, to an oligonucleotide present on a third proximity probe. A cassette oligo, that precisely spans the gap between the 3′ and 5′ ends of the first two oligonucleotides, is added, allowing the ends to be joined by enzymatic DNA ligation. The ligation products are then amplified by PCR and distinguished from unreacted probes. (See, for example, Fredriksson et al., (2002) Nat. Biotechnol. 20: 473-477, Gullberg et. al., (2004) Proc. Natl. Acad. Sci. U.S.A. 101: 8420-8424, Gullberg, et. al., (2003) Curr. Opin. Biotechnol. 14: 1-5, Pai et al., (2005) Nuc. Acids Res. 33: e162; US Patent Applications 2002/0064779; 2005/0003361).

### SUMMARY

Briefly described, embodiments of this disclosure, among others, encompass methods for the detection of a target analyte in a sample. The method makes use of the detection of at least two distinct sites on an analyte molecule, or the pairing of two distinct sites on two adjacent and contacting molecules. The distinct sites may be, but not necessarily, integral to the structure of a single molecule. For example, a detectable site may be the combination of one or more amino acids of a polypeptide sequence. The amino acids may be adjacent in the sequence or positioned near one another due to the three-dimensional structure of the polypeptide. It is also contemplated at least one of the detectable sites may be formed by a modification of a larger molecule. For example, a small molecule such as, but not limited to, a phosphate group, a glycosylation group, and the like, may be attached to the target analyte to form a distinct structure that may be recognizable and bound by a specific probe. The small molecule may be a tag such as, but not limited to, a dye or digoxin that may be recognizable by a probe.

One aspect of the present disclosure, therefore, encompasses embodiments of methods of detecting a target analyte, comprising the steps of: (i) obtaining a sample suspected of comprising a target analyte; (ii) contacting the sample with a first probe and a second probe, where the first probe and the second probe can each independently comprise a binding moiety capable of specifically binding to the target analyte or a tag thereon, and an oligonucleotide tail, said oligonucleotide tail comprising a PCR initiator region proximal to the target analyte binding moiety, a barcoding region uniquely associated with the target analyte binding moiety, and a connector-hybridizing region complementary to a region of a connector oligonucleotide, where the connector-hybridizing region is distal to the target analyte binding moiety, thereby capturing a target analyte in the sample; (iii) hybridizing a connector oligonucleotide to the connector-hybridizing regions of the first probe and the second probe; (iv) ligating the connector-hybridizing region of the first probe to the connector-hybridizing region of the second probe, thereby linking the oligonucleotide tails of the first probe and the second probe; (v) amplifying the region of the ligated oligonucleotide tails between the PCR initiator regions; (vi) hybridizing the amplification product with a substrate-immobilized oligonucleotide, where the substrate-immobilized oligonucleotide can comprise a first region complementary to the barcoding region uniquely associated with the target analyte binding moiety of the first probe and a second region complementary to the barcoding region uniquely associated the target analyte binding moiety of the second probe; (vii) contacting the product of step (v) with a nuclease capable of specifically digesting a single-strand DNA molecule or region thereof, where the single strand DNA has a non-base paired 3′ or a 5′ terminus; (viii) hybridizing a signaling oligonucleotide to the product of step (vi), where the signaling oligonucleotide comprises a nucleotide sequence complementary to a nucleotide sequence of the ligated connector-hybridizing region of the first probe, the connector-hybridizing region of the second probe, or a combination of the ligated connector-hybridizing region of the first probe, the connector-hybridizing region of the second probe, and further compromises a label; and (ix) detecting the label; thereby detecting the presence of the analyte in the sample.

In embodiments of this aspect of the disclosure, the target analyte can be selected from the group consisting of a peptide, a polypeptide, a protein, or a modified variant thereof.

In embodiments of this aspect of the disclosure, the binding moiety of each of the first probe and the second probe can be selected from the group consisting of an antibody, a fragment of an antibody, an aptamer, a peptide, a polypeptide, a biological receptor, and a ligand capable of binding to biomolecule.

In embodiments of this aspect of the disclosure, the sample can be contacted with a tag, thereby attaching a tag to the target analyte where the binding moiety of the first probe can specifically bind to a site of the target analyte and the binding moiety of the second probe can specifically bind to the tag. In these embodiments of this aspect of the disclosure, the tag can be selected from a dye, a fluorescent dye, and digoxin.

In embodiments of this aspect of the disclosure, the binding moiety of the second probe can specifically bind to a modification of a polypeptide.

In embodiments of this aspect of the disclosure, the modification of a polypeptide can be selected from the group consisting of a phosphorylated site, a glycosylated site, and a mutated site of the amino acid sequence of the polypeptide.

In embodiments of this aspect of the disclosure, the target analyte can be a combination of at least two polypeptides wherein the binding moiety of the first probe can specifically bind to a region of a first polypeptide and the binding moiety of the second probe can specifically bind to a region of a second polypeptide and where, in step (iii) hybridizing a connector oligonucleotide to the connector-hybridizing regions of the first probe and the second probe is when first polypeptide and the second polypeptide are complexed together.

In embodiments of this aspect of the disclosure, the nuclease capable of specifically digesting a single-strand DNA molecule or region thereof can be selected from the group consisting of: Rec J, Exonuclease II, Calf spleen phosphodiesterase, Exonuclease I (phosphodiesterase), Snake venom phosphodiesterase, and Exonuclease VII.

Another aspect of the present disclosure encompasses systems for detecting a target analyte, comprising: a first probe and a second probe, where the first probe and the second probe each independently can comprise a binding moiety capable of specifically binding to the target analyte or a tag thereon, and an oligonucleotide tail, said oligonucleotide tail comprising a first PCR initiator region proximal to the target analyte binding moiety, a barcoding region uniquely associated the target analyte binding moiety, and a connector-hybridizing region distal to the target analyte binding moiety; and a microarray, wherein the array comprises at least one oligonucleotide complementary to the barcoding region of the first probe and the barcoding region of the second probe.

In embodiments of this aspect of the disclosure, the binding moiety of the first probe can be attached to the 5′ terminus of the oligonucleotide tail, and the binding moiety of the second probe can be attached to the 3′ terminus of the oligonucleotide tail.

In embodiments of this aspect of the disclosure, the systems can further comprise an oligonucleotide complimentary to the PCR initiator region of the first probe and an oligonucleotide complimentary to the PCR initiator region of the second probe.

Yet another aspect of the present disclosure encompasses probes that can comprise a binding moiety capable of specifically binding to a target analyte or a tag thereon, and an oligonucleotide tail, said oligonucleotide tail comprising a PCR initiator region proximal to the target analyte binding moiety, a barcoding region uniquely associated the target analyte binding moiety, and a connector-hybridizing region, where the connector-hybridizing region is distal to the target analyte binding moiety, and where the probe is configured for use in the methods and systems according to the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates the array-based proximity ligation detection assay of the disclosure.

FIG. 2 illustrates in summary the detection of an array-bound single strand nucleic acid loop.

FIG. 3 schematically illustrates the detection of a labeled polypeptide by the array-based system of the disclosure.

FIG. 4 schematically illustrates the detection of a labeled polypeptide by the array-based system of the disclosure, after labeling of the target analyte(s) with a phosphate group by using a kinase.

FIG. 5 schematically illustrates the detection of the interaction of a specific polypeptide to proteins of a heterogeneous polypeptide library by the array-based system of the disclosure.

FIG. 6 diagrammatically illustrates the regions of a probe-analyte complex formed according to the procedures of the array-based system of the disclosure.

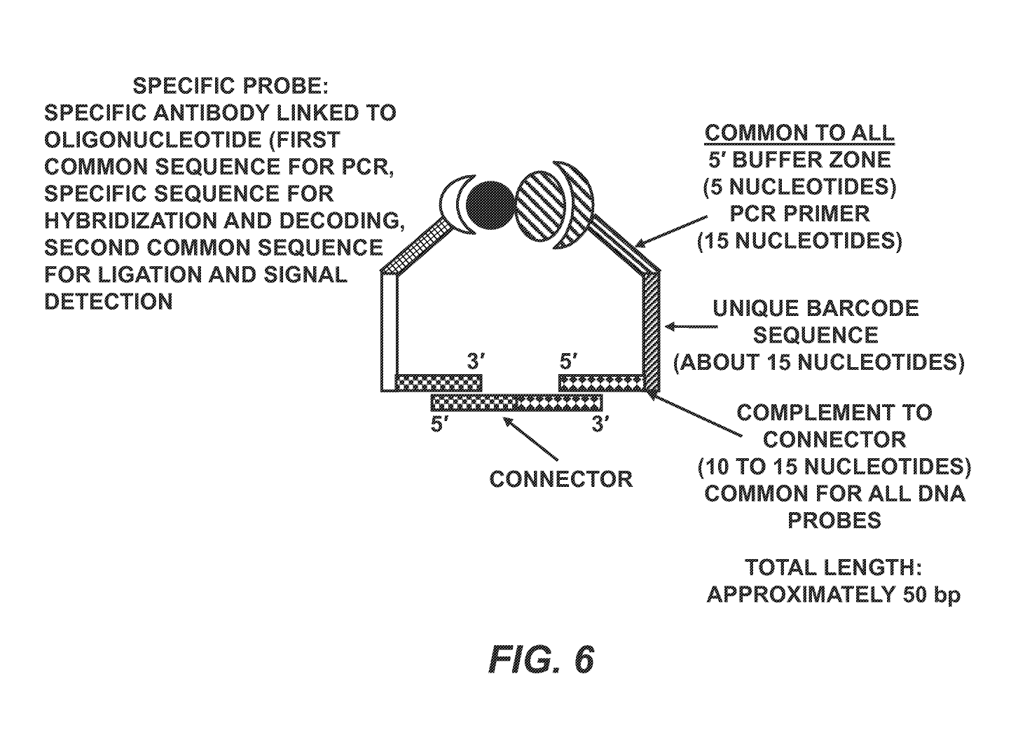

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### DEFINITIONS

In describing and claiming the disclosed subject matter, the following terminology will be used in accordance with the definitions set forth below.

The term “barcode” as used herein refers to an oligonucleotide of a predefined sequence, and which is associated with a specific target analyte binding moiety.

The terms “analyte” or “target analyte” as used herein refer to a biomolecule such as, but not limited to, a peptide, a polypeptide, a nucleic acid and the like to be detected in a sample. The analyte can be comprised of a member of a specific binding pair (sbp) and may be a ligand, which is monovalent (monoepitopic) or polyvalent (polyepitopic), preferably antigenic or haptenic, and is a single compound or molecule or a plurality of compounds or biomolecules, which share at least one common epitopic or determinant site. The analyte can be a part of a cell such as bacteria, a plant cell, an animal cell, either in a natural environment such as a tissue, or a cultured cell, a microorganism, e.g., bacterium, fungus, protozoan, or virus. If the analyte is monoepitopic, the analyte can be further modified, e.g. chemically, to provide one or more additional binding sites such as, but not limited to, a dye (e.g., a fluorescent dye), a polypeptide modifying moiety such as a phosphate group, a glycosidic group, and the like. In practicing the methods of the disclosure, the target analyte may have at least two binding sites. The polyvalent ligand analytes will normally be larger organic compounds, often of polymeric nature, such as polypeptides and proteins, polysaccharides, nucleic acids, and combinations thereof. Such combinations include components of bacteria, viruses, chromosomes, genes, mitochondria, nuclei, cell membranes and the like.

For the most part, the polyepitopic ligand analytes to which the subject invention can be applied will have a molecular weight of at least about 5,000, more usually at least about 10,000. In the polymeric molecule category, the polymers of interest will generally be from about 5,000 to about 5,000,000 molecular weight, more usually from about 20,000 to about 1,000,000 molecular weight; among the hormones of interest, the molecular weights will usually range from about 5,000 to about 60,000 molecular weight. The monoepitopic ligand analytes will generally be from about 100 to 2,000 molecular weight, more usually from about 125 to about 1,000 molecular weight.

The analyte may be a molecule found directly in a sample such as a body fluid from a host. The body fluid can be, for example, urine, blood, plasma, serum, saliva, semen, stool, sputum, cerebral spinal fluid, tears, mucus, and the like. The sample can be examined directly or may be pretreated to render the analyte more readily detectible.

The term “specific binding pair (sbp) member” as used herein refers to one of two different molecules that specifically bind to, and can be defined as complementary with, a particular spatial and/or polar organization of the other molecule. The members of the specific binding pair can be referred to as ligand and receptor (anti-ligand). These will usually be members of an immunological pair such as antigen-antibody, although other specific binding pairs such as biotin-avidin, enzyme-substrate, enzyme-antagonist, enzyme-agonist, drug-target molecule, hormones-hormone receptors, nucleic acid duplexes, IgG-protein A/protein G, polynucleotide pairs such as DNA-DNA, DNA-RNA, protein-DNA, lipid-DNA, lipid-protein, polysaccharide-lipid, protein-polysaccharide, nucleic acid aptamers and associated target ligands (e.g., small organic compounds, nucleic acids, proteins, peptides, viruses, cells, etc.), and the like are not immunological pairs but are included in the invention and the definition of sbp member. A member of a specific binding pair can be the entire molecule, or only a portion of the molecule so long as the member specifically binds to the binding site on the target analyte to form a specific binding pair.

The term “ligand” as used herein refers to any organic compound for which a receptor naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog will usually differ from the ligand by more than replacement of a hydrogen with a bond, which links the ligand analog to a hub or label, but need not. The ligand analog can bind to the receptor in a manner similar to the ligand. The analog could be, for example, an antibody directed against the idiotype of an antibody to the ligand.

The term “receptor” or “anti-ligand” as used herein refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, nucleic acid aptamers, avidin, protein A, barstar, complement component C1q, and the like. Avidin is intended to include egg white avidin and biotin binding proteins from other sources, such as streptavidin.

The term “specific binding” as used herein refers to the specific recognition of one molecule, of two different molecules, compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth.

The term “antibody” as used herein refers to an immunoglobulin which specifically binds to and is thereby defined as complementary with a particular spatial and polar organization of another molecule. The antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, IgY, etc. Fragments thereof may include Fab, Fv and F(ab′)₂, Fab′, scFv, and the like. In addition, aggregates, polymers, and conjugates of immunoglobulins or their fragments can be used where appropriate so long as binding affinity for a particular molecule is maintained.

The term “ligation” as used herein refers to the process of joining DNA molecules together with covalent bonds. For example, DNA ligation involves creating a phosphodiester bond between the 3′ hydroxyl of one nucleotide and the 5′ phosphate of another. Ligation is preferably carried out at 4-37° C. in the presence of a ligase enzyme. Examples of suitable ligases include Thermus thermophilus ligase, Thermus acquaticus ligase, E. coli ligase, T4 ligase, and Pyrococcus ligase.

The terms “specific”, “specifically”, or specificity” as used herein refer to the recognition, contact and formation of a stable complex between a molecule and another, together with substantially less to no recognition, contact and formation of a stable complex between the molecule and other molecules. Exemplary specific bindings are antibody-antigen interaction, cellular receptor-ligand interactions, polynucleotide hybridization, enzyme substrate interactions, etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence.

The terms “label” and “labeled molecule” as used herein as a component of a complex or molecule refer to a molecule capable of detection including, but not limited to, radioactive isotopes, fluorophores, chemoluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the term “labeling signal” as used herein refers to the signal emitted from the label that allows detection of the label, including but not limited to, fluorescence, chemolumiescence, production of a compound in outcome of an enzymatic reaction and the likes.

By “detectably labeled” is meant that a fragment or an oligonucleotide contains a nucleotide that is radioactive, or that is substituted with a fluorophore, or that is substituted with some other molecular species that elicits a physical or chemical response that can be observed or detected by the naked eye or by means of instrumentation such as, without limitation, scintillation counters, colorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, colorimetric, hybridization protection assay, and mass spectrometry

The term “aptamer” as used herein refers to an isolated nucleic acid molecule that binds with high specificity and affinity to a target, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is entirely fortuitous and plays no role whatsoever in the binding of an aptamer to its cognate target.

Aptamers must also be differentiated from the naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids, i.e., nucleic acid-binding proteins. Aptamers on the other hand are short, isolated, non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any target of interest including small molecules, carbohydrates, peptides, etc. For most targets, even proteins, a naturally occurring nucleic acid sequence to which it binds does not exist. For those targets that do have such a sequence, i.e., nucleic acid-binding proteins, such sequences will differ from aptamers as a result of the relatively low binding affinity used in nature as compared to tightly binding aptamers.

Aptamers are capable of specifically binding to selected targets and modulating the target's activity or binding interactions, e.g., through binding, aptamers may block their target's ability to function. The functional property of specific binding to a target is an inherent property an aptamer.

A typical aptamer is 6-35 kDa in size (20-100 nucleotides), binds its target with micromolar to sub-nanomolar affinity, and may discriminate against closely related targets (e.g., aptamers may selectively bind related proteins from the same gene family). Aptamers are capable of using commonly seen intermolecular interactions such as hydrogen bonding, electrostatic complementarities, hydrophobic contacts, and steric exclusion to bind with a specific target. Aptamers have a number of desirable characteristics for use as therapeutics and diagnostics including high specificity and affinity, low immunogenicity, biological efficacy, and excellent pharmacokinetic properties.

“DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

The terms “oligonucleotide” and “polynucleotide” as used herein refer to any polyribonucleotide or polydeoxribonucleotide that may be unmodified RNA or DNA or modified RNA or DNA. Thus, the term “polynucleotide” as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above. Typically, aptamers are single-stranded.

The term “glycosylation site” as used herein refers to a location on a polypeptide that has a glycosylation chain attached thereto. The “site” may be an amino acid side-chain, or a plurality of side-chains (either contiguous in the amino acid sequence of in cooperative vicinity to one another to define a specific site associated with at least one glycosylation chain).

The term “hybridization” as used herein refers to the process of association of two nucleic acid strands to form an anti-parallel duplex stabilized by means of hydrogen bonding between residues of the opposite nucleic acid strands.

“Hybridizing” and “binding”, with respect to polynucleotides, are used interchangeably. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The terms “complementarity” or “complementary” as used herein for the purposes of the specification or claims refers to a sufficient number in the oligonucleotide of complementary base pairs in its sequence to interact specifically (hybridize) with the target nucleic acid sequence to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

The term “DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

By the terms “enzymatically amplify” or “amplify” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); Qβ replicase amplification (OBRA); self-sustained replication (3SR); and NASBA (nucleic acid sequence-based amplification), which can be performed on RNA or DNA as the nucleic acid sequence to be amplified.

By “immobilized on a solid support” is meant that a fragment, primer or oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this invention.

A “primer” is an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

### DESCRIPTION

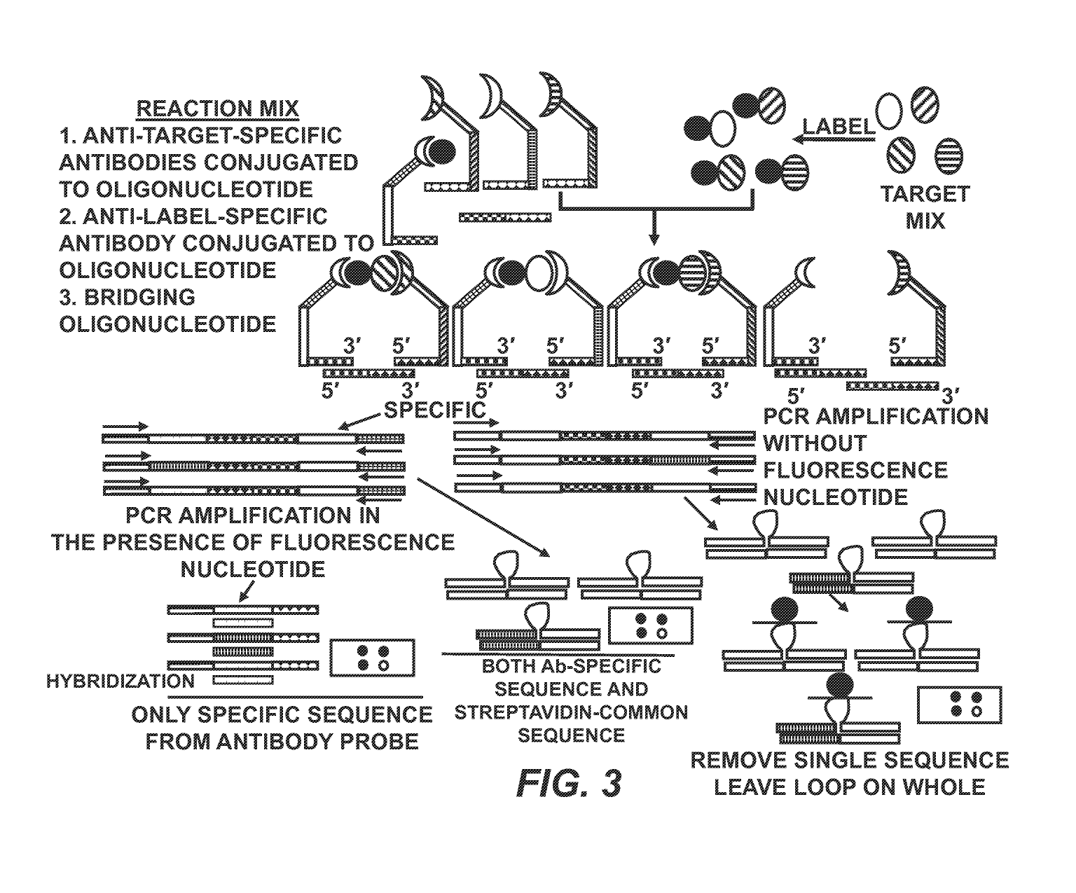

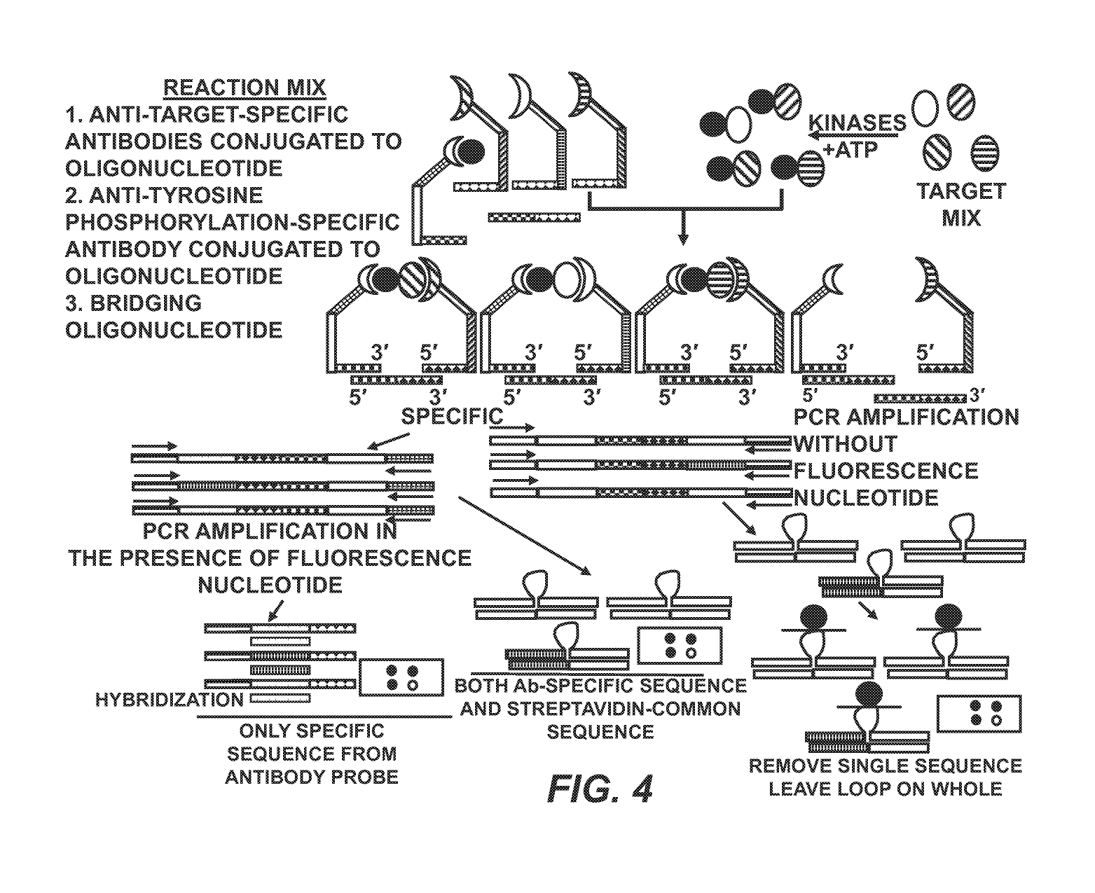

The present disclosure provides methods for the detection of a target analyte in a sample. The method makes use of the detection of at least two distinct sites on an analyte molecule, or the pairing of two distinct sites on two adjacent but contacting molecules. The distinct sites may be, but are not necessarily, due to the structure of the target analyte molecule. For example, a detectable site may be the combination of one or more amino acids of a polypeptide sequence. The amino acids may be adjacent in the sequence, or positioned near one another due to the three-dimensional structure of the polypeptide. It is also contemplated that at least one of the detectable sites may be formed by a modification of the larger molecule. For example, a small molecule such as, but not limited to, a phosphate group, a glycosylation group, and the like may be attached to the target analyte to form a distinct structure that may be recognizable and bound by a specific probe. The small molecule may be a tag such as, but not limited to, a dye or digoxin that may be recognizable by a probe. As shown in FIG. 3, the small molecule tag may be attached to potential analytes by such as an enzyme reaction. For example, but not limiting, a phosphate group may be attached to a target analyte by a kinase, as shown in FIG. 4.

The probes of the methods herein disclosed each comprise a moiety capable of recognizing and specifically binding to a distinct site of the target analyte. This moiety is linked to an oligonucleotide tail that preferably comprises three regions. The first region, proximal to the analyte binding moiety is a nucleotide sequence of sufficient length and sequence uniqueness to be useful as a complementary binding site for an oligonucleotide amplification primer. The next and adjacent region is a nucleotide sequence encoding a barcode that is uniquely associated with the analyte-binding moiety of the probe. A synthesis of barcode oligonucleotides is described, for example, in Xu et al., (2009) Proc. Natl. Acad. Sci. U.S.A. 106: 2289-2294, incorporated herein by reference in its entirety. The third region of the oligonucleotide tail is a nucleotide sequence complementary to a sequence of a connector oligonucleotide.

Detection of an analyte by the methods of the disclosure requires the use of a first probe and a second probe, each probe specifically recognizing regions of the target analyte. In the first probe, the oligonucleotide tail is linked to the analyte binding moiety through the 5′ terminus of the oligonucleotide. For the second probe, the oligonucleotide tail is linked to the analyte binding moiety through the 3′ terminus of the oligonucleotide.

The target analyte binding moiety may be any molecule that can specifically recognize and bind to a site on the analyte. It is contemplated that a binding moiety may be, but is not limited to, an antibody (IgA IgE, IgG, IgM, or IgY) of a fragment thereof that has retained analyte binding activity. A target-specific aptamer, a small ligand molecule, or a receptor protein able to specifically bind to a region of the target may also be suitable binding moieties. Furthermore, a polypeptide known to form a complex with the target polypeptide analyte under natural conditions may be used as the analyte-specific moiety of a probe.

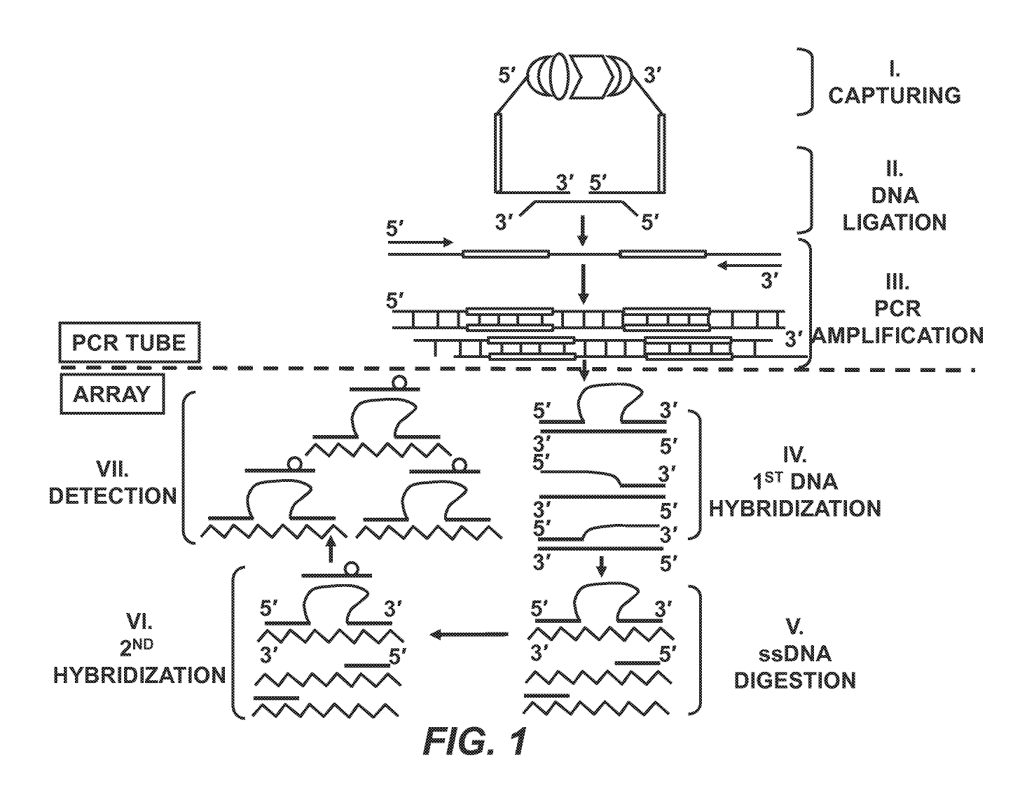

The initial step in the methods of the disclosure, therefore, requires that a sample suspected of including an analyte of interest be admixed with at least a first probe and a second probe as described above, whereupon the probes can bind to their respective sites on the target analyte. By so doing, their oligonucleotide tails are brought into close proximity. The sample is then incubated under conditions conducive to oligonucleotide annealing and with a high molar concentration of a connector oligonucleotide, one region of which complements the free end of the oligonucleotide tail of the first probe, and the remainder of the connector sequence complements the free end of the tail of the second probe. Accordingly, the terminal base of one tail is adjacent to the other, and positioned to allow for a ligation reaction to link one tail to the other, as shown, for example in FIG. 1.

Following ligation of the two oligonucleotide tails, the entire construct extending from, but not including, the analyte binding moieties can be amplified by well known methods and using primers complementing the tail regions immediately adjacent to the two analyte binding moieties.

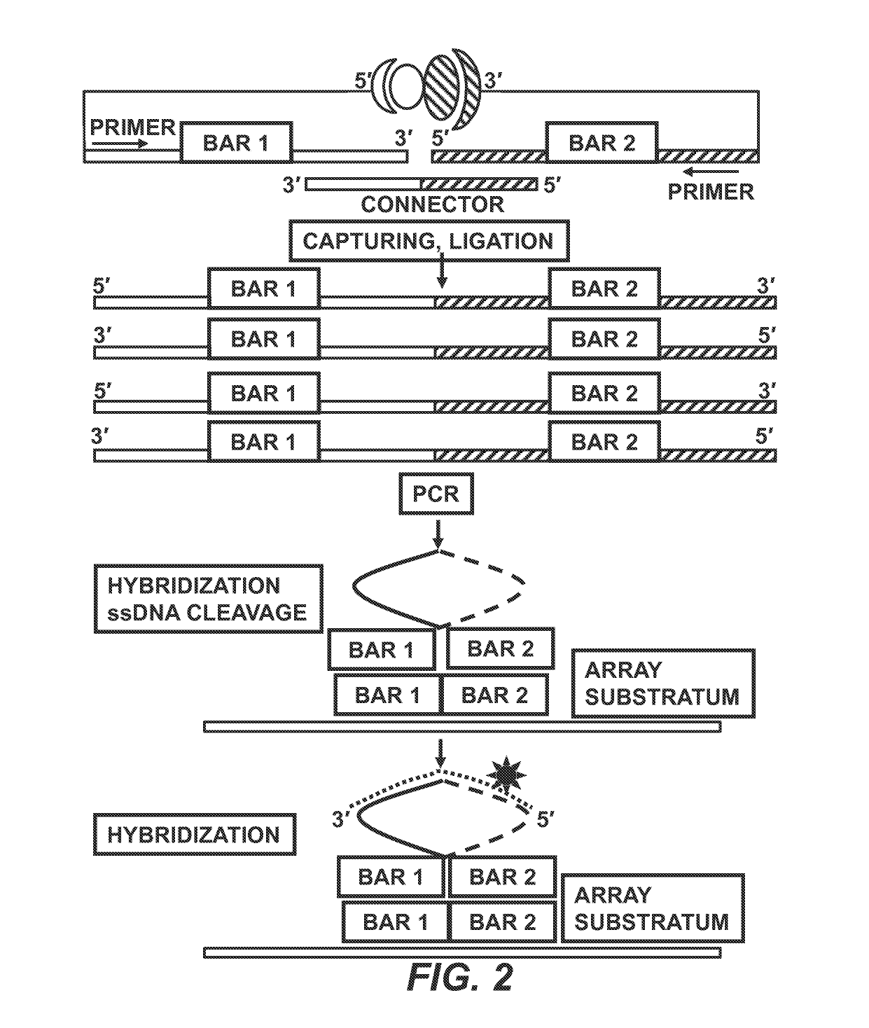

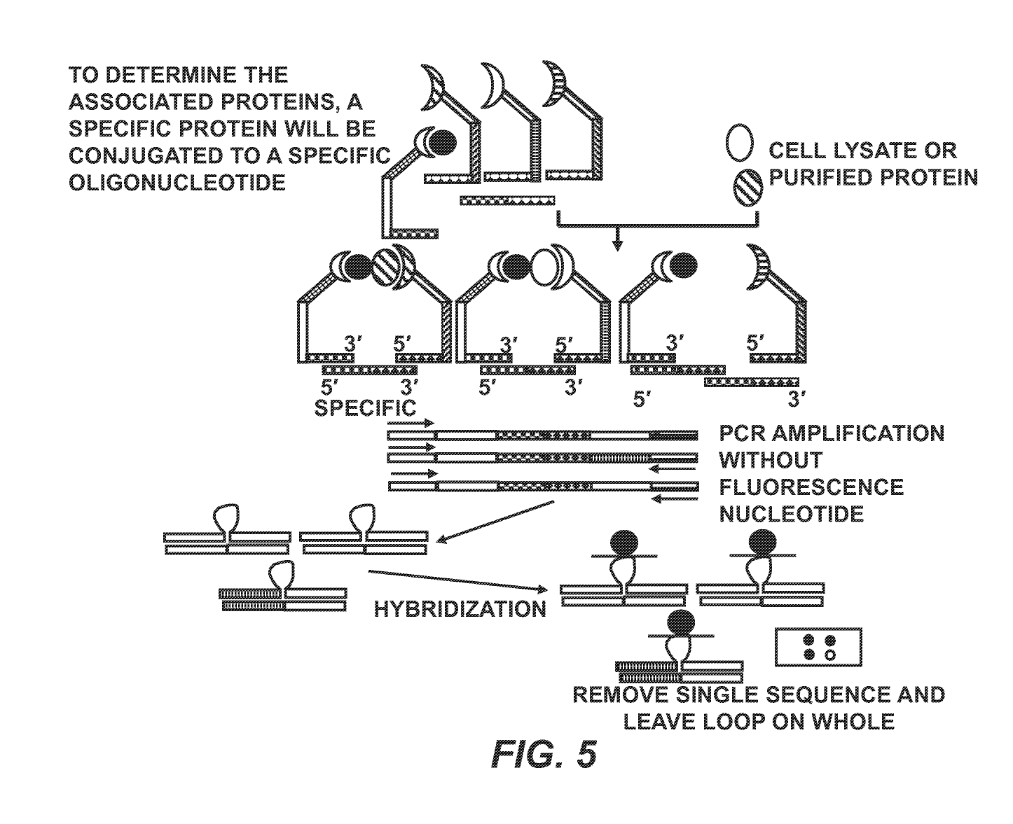

PCR products can then be hybridized to an oligonucleotide array, wherein each array spot comprises a target oligonucleotide comprising two barcode sequences, one complementary to the barcode of the first probe, and the other complementary to the barcode of the second probe. Where hybridization occurs, as shown schematically in FIGS. 1-5, the tail regions that complemented the connector oligonucleotide now form a single-strand loop structure, but with no free terminus. On the other hand, where a PCR product has barcode regions, only one of which binds to a specific array site, the unbound portion of the PCR product will be a single-strand sequence with a free terminus.

Specificity of the array analysis requires the next step of the method, which is to treat the array with an exonuclease that is single strand specific (the exonuclease activity will not digest the loop structure that formed when both barcode regions of a PCR product have bound to an array spot). The exonuclease activity, therefore, removes the unbound barcode region and the connector specific region adjacent thereto. Following the nuclease reaction, the only single strand nucleotide region remaining associated with the array are the loops formed where both barcode regions of the PCR reaction products have hybridized to a single array target.

The remaining single strand loops are then detected by hybridizing the array with a labeled oligonucleotide probe, the sequence of which complements at least part of the connector sequence. Finally, the presence of the label is detected, thereby identifying the presence of the analyte target.

The methods of the disclosure, therefore, provide for the array detection of the products of proximity ligation reactions. By incorporating the single strand nuclease digestion step, the specificity of the assay is ensured by eliminating false positive results, focusing instead on a detectable result only where two probes bind to the same analyte and then only to the spot on the array having an oligonucleotide specific for the combination of the two barcodes.

It is contemplated that the method of the present disclosure can be useful for a several types of analyses. For example, but not intended to be limiting, embodiments of such assays may include:

(a) protein-protein interaction: The first probe specifically recognizes and binds to a site on a first polypeptide, and the second probe recognizes and binds to a site on a second polypeptide. The initial proximity ligation reaction will only occur if the first and second polypeptides are complexed. By using a single first probe specific for a particular first target polypeptide and a plurality of second probes specific to a plurality of polypeptides, it is possible to detect and identify the polypeptide that complexes with the first. Alternatively, a plurality of first and second probes together can be used to identify which, of a library of polypeptides or peptides can complex with other members of the library.

(b) protein expression: a single target analyte polypeptide can be identified from a mixture of polypeptides by using in the methods of the disclosure, a first probe and a second probe wherein each probe can independently recognize and bind to sites on the same protein.

(c) protein modification: the detection of a modified polypeptide from a population of unmodified polypeptides may be identified by the methods of the disclosure, where the first probe specifically recognizes a site on the target a polypeptide analyte, and the second probe can specifically recognize and bind to a modifying moiety attached to the polypeptide. It is contemplated that the modifying group can be such as, but is not limited to, a phosphate group, a glycosidic group, a modified amino acid or a mutated site within the polypeptide.

(d) nucleic acid-polypeptide interaction: the methods of the disclosure may indentify a nucleic acid, DNA or RNA, able to recognize and bind to a target polypeptide. In these methods, the first probe may recognize and bind to a site of the target polypeptide, and the binding moiety of the second probe is an oligonucleotide suspected of binding to the polypeptide.

(e) protein-small molecule interaction; the methods of the disclosure may indentify a small molecule able to recognize and bind to a target polypeptide. In these methods, the first probe may recognize and bind to a site of the target polypeptide, and the second probe can specifically recognize and bind to a small molecule of ligand suspected of binding to the polypeptide.

One aspect of the present disclosure, therefore, encompasses embodiments of methods of detecting a target analyte, comprising the steps of: (i) obtaining a sample suspected of comprising a target analyte; (ii) contacting the sample with a first probe and a second probe, where the first probe and the second probe can each independently comprise a binding moiety capable of specifically binding to the target analyte or a tag thereon, and an oligonucleotide tail, said oligonucleotide tail comprising a PCR initiator region proximal to the target analyte binding moiety, a barcoding region uniquely associated with the target analyte binding moiety, and a connector-hybridizing region complementary to a region of a connector oligonucleotide, where the connector-hybridizing region is distal to the target analyte binding moiety, thereby capturing a target analyte in the sample; (iii) hybridizing a connector oligonucleotide to the connector-hybridizing regions of the first probe and the second probe; (iv) ligating the connector-hybridizing region of the first probe to the connector-hybridizing region of the second probe, thereby linking the oligonucleotide tails of the first probe and the second probe; (v) amplifying the region of the ligated oligonucleotide tails between the PCR initiator regions; (vi) hybridizing the amplification product with a substrate-immobilized oligonucleotide, where the substrate-immobilized oligonucleotide can comprise a first region complementary to the barcoding region uniquely associated with the target analyte binding moiety of the first probe and a second region complementary to the barcoding region uniquely associated the target analyte binding moiety of the second probe; (vii) contacting the product of step (v) with a nuclease capable of specifically digesting a single-strand DNA molecule or region thereof, where the single strand DNA has a non-base paired 3′ or a 5′ terminus; (viii) hybridizing a signaling oligonucleotide to the product of step (vi), where the signaling oligonucleotide comprises a nucleotide sequence complementary to a nucleotide sequence of the ligated connector-hybridizing region of the first probe, the connector-hybridizing region of the second probe, or a combination of the ligated connector-hybridizing region of the first probe, the connector-hybridizing region of the second probe, and further compromises a label; and (ix) detecting the label; thereby detecting the presence of the analyte in the sample.

In embodiments of this aspect of the disclosure, the target analyte can be selected from the group consisting of a peptide, a polypeptide, a protein, or a modified variant thereof.

In embodiments of this aspect of the disclosure, the binding moiety of each of the first probe and the second probe can be selected from the group consisting of an antibody, a fragment of an antibody, an aptamer, a peptide, a polypeptide, a biological receptor, and a ligand capable of binding to biomolecule.

In embodiments of this aspect of the disclosure, the sample can be contacted with a tag, thereby attaching a tag to the target analyte where the binding moiety of the first probe can specifically bind to a site of the target analyte and the binding moiety of the second probe can specifically bind to the tag. In these embodiments of this aspect of the disclosure, the tag can be selected from a dye, a fluorescent dye, and digoxin.

In embodiments of this aspect of the disclosure, the binding moiety of the second probe can specifically bind to a modification of a polypeptide.

In embodiments of this aspect of the disclosure, the modification of a polypeptide can be selected from the group consisting of a phosphorylated site, a glycosylated site, and a mutated site of the amino acid sequence of the polypeptide.

In embodiments of this aspect of the disclosure, the target analyte can be a combination of at least two polypeptides wherein the binding moiety of the first probe can specifically bind to a region of a first polypeptide and the binding moiety of the second probe can specifically bind to a region of a second polypeptide and where, in step (iii) hybridizing a connector oligonucleotide to the connector-hybridizing regions of the first probe and the second probe is when first polypeptide and the second polypeptide are complexed together.

In embodiments of this aspect of the disclosure, the nuclease capable of specifically digesting a single-strand DNA molecule or region thereof can be selected from the group consisting of: Rec J, Exonuclease II, Calf spleen phosphodiesterase, Exonuclease I (phosphodiesterase), Snake venom phosphodiesterase, and Exonuclease VII.

Another aspect of the present disclosure encompasses systems for detecting a target analyte, comprising: a first probe and a second probe, where the first probe and the second probe each independently can comprise a binding moiety capable of specifically binding to the target analyte or a tag thereon, and an oligonucleotide tail, said oligonucleotide tail comprising a first PCR initiator region proximal to the target analyte binding moiety, a barcoding region uniquely associated the target analyte binding moiety, and a connector-hybridizing region distal to the target analyte binding moiety; and a microarray, wherein the array comprises at least one oligonucleotide complementary to the barcoding region of the first probe and the barcoding region of the second probe.

In embodiments of this aspect of the disclosure, the binding moiety of the first probe can be attached to the 5′ terminus of the oligonucleotide tail, and the binding moiety of the second probe can be attached to the 3′ terminus of the oligonucleotide tail.

In embodiments of this aspect of the disclosure, the systems can further comprise an oligonucleotide complimentary to the PCR initiator region of the first probe and an oligonucleotide complimentary to the PCR initiator region of the second probe.

Yet another aspect of the present disclosure encompasses probes that can comprise a binding moiety capable of specifically binding to a target analyte or a tag thereon, and an oligonucleotide tail, said oligonucleotide tail comprising a PCR initiator region proximal to the target analyte binding moiety, a barcoding region uniquely associated the target analyte binding moiety, and a connector-hybridizing region, where the connector-hybridizing region is distal to the target analyte binding moiety, and where the probe is configured for use in the methods and systems according to the present disclosure.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

### Conjugation of Antibodies with Oligonucleotides

The paired antibodies recognize the same protein at the different epitopes and this can be identified through well-documented immunoassay. cDNA conjugated Ab can be generated by simple linking of thiol-cDNA to sulfo-GMBS-treated antibody. The effect of conjugation on the ability of the antibody to bind antigen will be determined by ELISA assay. Methods: using the kit from Solulink Conjugation Company. The SoluLink Protein-Oligo Conjugation Kit™ uses a bioconjugation method to prepare protein-oligonucleotide conjugates in three steps: (i) modification of the antibody protein with S-HyNic crosslinker (succinimidyl 6-hydrazinonicotinate acetone hydrazone; (ii) modification of the oligonucleotide with 4FB; and (iii) conjugation of the two modified biomolecules.

(a) Desalt/Buffer Exchange of the antibody: antibodies must be completely desalted into modification buffer (100 mM phosphate, 150 mM NaCl, pH 7.4).

(b) Modify the antibody with S-HyNic according to the manufacture's instruction.

(c) Desalt the HyNic-modified IgG into conjugation buffer (100 mM phosphate, 150 mM NaCl, pH 6.0).

(d) Desalt the oligonucleotide into nuclease free water using a 5K MWCO VivaSpin diafiltration apparatus.

(e) Modify the amino oligonucleotide with 4FB according to the manufacture's instruction.

(f) Mix the HyNic-modified protein with the 4FB-modified oligonucleotide (2 equivalents of oligo/conjugated oligo desired)

(g) Add 1/10 volume 10× TurboLink Catalyst Buffer to the conjugation solution.

(h) Incubate the mixture at room temperature for 2 hr (the conjugation reaction can be ‘visualized’ by removing an aliquot and analyzing by gel electrophoresis or spectrophotometrically on a NanoDrop spectrophotometer by determining the absorbance at A354 due to the formation of the chromophoric conjugate bond).

(i) Desalt the conjugate with column.

### Alternative Method 1 (Self-Assembly Strategy)

(a) Forming sptreptavidin-oligonucleotide conjugates from streptavidin and biotinylated oligunucleotides.

(b) Conjugating biotin-antibody with streptavidin-oligonuleotide to self assemble proximity probes, according to the methods of Darmanis et al., (2007) BioTechniques 43: 443-450, incorporated herein by reference in its entirety.

### Alternative Method 2

(a) Conjugating antibody with sulfo-GMBS (Sulfo-GMBS (N-[g-Maleimidobutyryloxy]sulfosuccinimide ester).
(b) Removing untreated sulfo-GMBS by chromatography over a PD-10 column.
(c) The sulfo-GMBS-activated antibody and 5′ thiol DNA are conjugated together.
(d) Antibody conjugated to DNA is purified by anion exchange chromatography on superdex-200.

### Example 2

### Capturing of Target Molecules by Paired Antibodies and DNA Ligation

(i) Diluted the test samples (containing target molecules) with Buffer A;
(ii) Take a 0.2 mL thin-well PCR tubes. For 50 μL reaction, add: 1 μL of test sample (in Buffer A); 54 of 20 pM paired oligoDNA-conjugated antibodies (in Buffer B); 45 μL of mixture (premixed in 0.5 mL tube as following): 1× Buffer C; 0.4 units T4 DNA ligase; 400 nM connector oligonucleotide; 0.2 mM dNTPs; 0.5 μM Forward primer; 0.5 μM Reverse primer; 1.5 units Taq DNA polymerase;
(iii) Place tube on PCR Cycler; and
(iv) Incubation at 25° C. for 5 min to allow: (a) the paired antibodies to specifically capture the target molecules; (b) the oligonucleotides and the connector to anneal with the proximity 5′ and 3′ ends of oligonucleotide conjugated with antibodies; (c) T4 DNA ligase joins these two ends.

### Example 3

### PCR Amplification

Step 1. Initial denaturation: 95° C., 5 min

### Step 2. Denaturation: 95° C., 15 sec

### Step 3. Annealing: 55° C., 20 sec

### Step 4. Elongation: 72° C., 45 sec

Repeat Steps 2-4 for 35 times

Step 5. Final elongation: 72° C., 3 min

### Example 4

### Hybridization of PCR products with arrayed barcode oligonucleotides

(i) Heat PCR product at 95° C. for 5 min on PCR Cycler to denature dsDNA PCR products to ssDNA.

(ii) Immediately chill the tube on ice for 3 min.

(iii) Spin the tube for seconds.

(iv) Prehybridize array slide with 100 μL of 1×DNA hybridization buffer at 42° C. for 20 min.

(v) Remove the 1×DNA hybridization buffer.

(vi) Dilute the denatured PCR products with 1×DNA hybridization buffer to 100 μL, and apply it onto array slide.

(vii) Incubate at 42° C. for 1 hr in the covered wet chamber.

(viii) Wash with 1× washing buffer (1× SSC, 0.1% SDS), 3 times.

(ix) Wash with 1× exonuclease reaction buffer once.

### Example 5

### Free Single Strand DNA Cleavage

(i) Add 50 μL 1× exonuclease reaction buffer.

(ii) Add, e.g. exonuclease VII. (see Table 1 for exonuclease selection)

TABLE 1
Potential ssDNA Exonuclease for PLA

| | | MW | Enzyme | Hairpin | |
|---|---|---|---|---|---|
| Enzyme | Hydrolysis | (kDa) | Source | Resistant | Reference |
| Rec J | 5′-3′ | 60 | E. coli | Yes | Lovett et al. PNAS. |
| | | | | | (1989), 86: 2627 |
| Exonuclease II | 5′-3′ | 500 | S. cerevisiae | Yes | Villadsen et al. JBC. |
| | | | | | (1982) 257: 8177 |
| Calf spleen | 5′-3′ | | Calf | Yes | Bartolini et al. J Am |
| phosphodiesterase | | | spleen | | Soc Mass Spectrom. |
| | | | | | (1999) 10: 521 |
| Exonuclease I | 3′-5′^{b} | | E. coli | No | Lehman et al. JBC. |
| (phosphodiesterase)^{a} | | | | | (1964), 239: 2628 |
| Snake venom | 3′-5′ | | Snake | Yes | Bartolini et al. J Am |
| phosphodiesterase | | | venom | | Soc Mass Spectrom. |
| | | | | | (1999), 10, 521 |
| Exonuclease VII | 5′-3′ and | | E. coli | No | Chase et al. JBC. |
| | 3′-5′ | | 10.5, 54 kDa | | 1974, 249: 4553 |

^{a}dsDNA is attacked 40,000 times less than ssDNA
^{b}Need a free terminal 3′-OH

(iii) Incubate at 37 μL for 30 min according to the manufacture's instruction to cleave the single strand of DNA from 5′ to 3′ ends and 3′ to 5′ ends.

(iv) Wash with water or 1× PBS, 3 times.

### Example 6

### Fluorescence-Labeled DNA Probe Hybridization

(i) Prehybridize the slide array with 100 μL of 1× DNA hybridization buffer at 42° C. for 20 min.

(ii) Remove the DNA hybridization buffer.

(iii) Incubate the array slide with 50 μL fluorescence-labeled oligonucleotide probe (diluted with DNA hybridization buffer) at 42° C. for 1 hr.

(iv) Wash with 1× washing buffer, 3 times.

(v) Wash with water, twice.

(vi) Decant the excess water completely.

(vii) Shortly dry slide at ambient temperature in the dark.

(viii) Store at 4° C. or −20° C. protected from light and humid.

### Example 7

### Signal Detection

(i) Scan the slide to read the fluorescence signal with the specific excitation wavelength.
(ii) Decode and analyze the data.

1-13. (canceled)

14. A system for detecting a target protein or polypeptide analyte, comprising:
(a) at least one probe pair consisting of a first probe and a second probe, wherein each probe comprises: (i) an antibody or a fragment thereof capable of specifically binding to a target protein or polypeptide analyte or a tag thereon, and (ii) an oligonucleotide tail, said oligonucleotide tail comprising a PCR initiator region proximal to the antibody or a fragment thereof, a barcoding region uniquely associated with the antibody or a fragment, and a connector-hybridizing region distal to the antibody or a fragment thereof, wherein the antibody or a fragment thereof of the first probe is conjugated to the 5′ terminus of a first oligonucleotide tail and the antibody or fragment thereof of the second probe is conjugated to the 3′ terminus of a second oligonucleotide tail;
(b) a connector oligonucleotide complementary to the barcoding region of the first probe and the barcoding region of the second probe;
(c) an oligonucleotide complimentary to the PCR initiator region of the first probe; and
(d) an oligonucleotide complimentary to the PCR initiator region of the second probe,
wherein the antibody or fragment thereof of the first probe and the antibody or fragment thereof of the second probe each independently and specifically binds to a target protein or polypeptide analyte and sufficiently close to one another to allow the oligonucleotide tails of the analyte-bound first and second probes to simultaneously hybridize to the connector oligonucleotide.

15. The system of claim 10, comprising a plurality of probe pairs, wherein each probe pair selectively binds to a target protein or polypeptide analyte of a plurality of such target analytes.

16. The system of claim 10, wherein the connector oligonucleotide complementary to the barcoding regions of the first and the second probes of each probe pair are arrayed on a substrate.

17. A probe consisting of:
(i) an antibody or a fragment thereof capable of specifically binding to a target protein or polypeptide analyte or a tag thereon; and
(ii) an oligonucleotide tail conjugated to a terminus of the antibody or fragment thereof, said oligonucleotide tail comprising a PCR initiator region proximal to the antibody or a fragment thereof, a barcoding region uniquely associated with the antibody or a fragment, and a connector-hybridizing region distal to the antibody or a fragment thereof.

18. A method comprising:
(i) capturing from a sample a target protein or polypeptide analyte having two probe-binding sites with a first probe and a second probe, wherein each probe binds to one but not the other of the two probe-binding sites, and wherein each probe independently consists of:
(a) an antibody or a fragment thereof capable of specifically binding to a target protein or polypeptide analyte or a tag thereon; and
(b) an oligonucleotide tail conjugated to a terminus of the antibody or fragment thereof, said oligonucleotide tail comprising a PCR initiator region proximal to the antibody or a fragment thereof, a barcoding region uniquely associated with the antibody or a fragment, and a connector-hybridizing region distal to the antibody or a fragment thereof,
wherein the antibody or fragment thereof of the first probe and the antibody or fragment thereof of the second probe each independently and specifically binds to a target protein or polypeptide analyte and sufficiently close to one another to allow the oligonucleotide tails of the analyte-bound first and second probes to simultaneously hybridize to the connector oligonucleotide;
(ii) hybridizing a connector oligonucleotide to the connector-hybridizing regions of each of the first and second probes bound to the analyte;
(iii) ligating the termini of the oligonucleotide tails hybridized to the connector oligonucleotide;
(iv) amplifying the entire region of the ligated oligonucleotide tails between the PCR initiator regions thereof, wherein the amplification product is a double-stranded nucleic acid consisting of the PCR initiator regions, the barcoding regions of the ligated oligonucleotide tails of the first and second probes, and the connector hybridizing regions located between said barcoding regions;
(v) hybridizing a strand of the amplification product of step (iv) to a substrate-immobilized oligonucleotide consisting of a region complementary to the barcoding region of the first probe and a region complementary to the barcoding region of the second probe;
(vi) incubating the product of step (vi) with a single-stranded DNA-specific exonuclease;
(vii) hybridizing a signaling oligonucleotide to the product of step (vi), the signaling oligonucleotide consisting of a detectable label and a nucleotide sequence complementary to connector-hybridizing regions of the ligated oligonucleotide tails to generate a labeled substrate-immobilized hybridization product only when the analyte is present in the sample;
(viii) removing signaling oligonucleotides not hybridized to the product of step (vii); and
(ix) determining whether the sample included the analyte by detecting a substrate-immobilized labeled hybridization product from step (viii).
